(19) United States
(12) Patent Application Publication
(10) Pub. No.: US 2024/0084351 A1
(43) Pub. Date: Mar. 14, 2024

(54) IMPROVED METHOD FOR REGENERATING REDUCED FORMS OF ENZYME COFACTORS

(71) Applicant: BIOCHEMINSIGHTS, INC., Malvern, PA (US)

(72) Inventors: David Dodds, Malvern, PA (US); William Armiger, Malvern, PA (US); Benedict Schwegler, Malvern, PA (US)

(73) Assignee: BIOCHEMINSIGHTS, INC., Malvern, PA (US)

(21) Appl. No.: 18/263,629

(22) PCT Filed: Jan. 31, 2022
(86) PCT No.: PCT/US2022/014640

§ 371 Date: Jul. 31, 2023

Related U.S. Application Data
(60) Provisional application No. 63/143,918, filed on Jan. 31, 2021.

(51) Int. Cl. C12P 19/36; C12N 9/02; C25B 3/05; C25B 3/25; C25B 11/032

(52) U.S. Cl. CPC C12P 19/36; C12N 9/0067; C25B 3/05; C25B 3/25; C25B 11/032

(57) ABSTRACT

Provided herein are composition and process for using a device for the reduction of the oxidized state of phosphorylated or non-phosphorylated nicotinamide adenine dinucleotide to the reduced state, using a catalyst to enable the reduction of the oxidized form of the phosphorylated or non-phosphorylated nicotinamide adenine dinucleotide by hydrogen, and methods for providing the hydrogen.

### CROSS REFERENCE TO RELATED APPLICATIONS

This application claims priority to and the benefit of U.S. Provisional Application No. 63/143,918 filed Jan. 31, 2021, the entire disclosure of which is incorporated herein by reference.

### TECHNICAL FIELD

The present disclosure relates generally to the use of biologically mediated reactions that alter the oxidation state of compounds, and specifically the oxidation state of carbon atoms in a given chemical compound.

More specifically, the present disclosure relates to an improved method and system of reducing the oxidized form of enzyme cofactors, most especially the oxidized form of the nicotinamide adenine dinucleotide cofactor, to their reduced states, most especially the reduced form of the nicotinamide adenine dinucleotide cofactor.

### BACKGROUND

The use of enzymes which perform a reduction, and are capable of reducing a provided substrate to a desired reduced product, have been widely reported. (Dodds et al, J. Am. Chem. Soc., (1988) 110(2), 577-583; Dodds et al, Proceedings Chiral Europe '95. London, (1995), 55-62; A. Liese et al, Appl Microbiol Biotechnol (2007) 76:237-248; Liese, A.; 2nd ed. Enzyme Catalysis in Organic Synthesis, (2002), 3, 1419-1459; Kula, M. R.; Kragl, U. Stereoselect. Biocatal. (2000), 839-866; Chartrain, M.; Greasham, R.; Moore, J.; Reider, P.; Robinson, D.; Buckland, B. J. Mol. Catal. B: Enzym. (2001), 11, 503-512; Patel, R. N. Enzyme Microb. Technol. (2002), 31, 804-826; Patel, R. N. Adv. Appl. Microbiol. (2000), 47, 33-78; Patel, R. N.; Hanson, R. L.; Banerjee, A.; Szarka, J. Am. Oil Chem. Soc. (1997), 74, 1345-1360; Hummel, W. Adv. Biochem. Eng. Biotechnol. (1997), 58, 145-184; Whitesides et al, Appl. Biochem. and Biotech. (1987) 14, 147-197; Whitesides et al, Biotechnology and Genetic Engineering Reviews, Vol. 6. September 1988).

To accomplish such a reduction of a provided substrate by enzyme catalysis, electrons must be provided to the reaction, and are electrically balanced by protons. In biological systems, both in vivo and in vitro, these electrons (and the balancing protons), are generally termed “reducing equivalents”, and are transported to enzymes requiring the reducing equivalents by small molecules generally termed “cofactors” (Andrew W. Munro, Kirsty J. McLean; “Electron Transfer Cofactors”, doi.org/10.1007/978-3-642-16712-6_41).

A need exists for an improved method and system of reducing the oxidized form of enzyme cofactors.

### BRIEF DESCRIPTION OF THE DRAWINGS

FIG. 1 illustrates, in a schematic, a system of providing a chamber or vessel, in which hydrogen gas is presented on the first side of a membrane, with a catalyst on the second side of the membrane, enabling the reduction of the cofactor, the cofactor being present in a process stream that enters chamber or vessel on the second side of the membrane.

FIG. 2 illustrates, in a schematic, a system of providing hydrogen by means of water electrolysis, with a catalyst enabling the reduction of the cofactor using the newly formed hydrogen, such catalyst being attached to the surface of the cathode, or contained in the porous structure of the cathode.

### SUMMARY

Provided herein, in one aspect, is a system for providing NAD(P)H₂ reducing equivalents to an in vitro system, comprising:
a chamber configured to receive hydrogen and to contain a liquid phase, said liquid phase comprising a cofactor that can be reduced by hydrogen from an oxidized form to a reduced form, for use in a desired redox reaction performed by an in vitro process;
a catalyst which enhances a reduction rate of the oxidized form by the hydrogen, thus forming the reduced form;
a process stream comprising a substrate to be transformed via the desired redox reaction which oxidizes the reduced form of the cofactor with concomitant production of a desired product; and
optionally, a membrane enabling containment within the chamber, the cofactor in the oxidized and/or reduced forms, the catalyst, and the process stream.

In some embodiments, the catalyst is a hydrogenase enzyme.

In some embodiments, the system further includes attachment of the catalyst to the membrane surface by physical or chemical methods.

In some embodiments, the structure of the chamber or vessel allowing the positioning of the catalyst immediately proximal to the source of molecular hydrogen such that the hydrogen is presented to the catalyst as molecular hydrogen dissolved in the process medium.

In some embodiments, the system further includes containment of the catalyst in the optionally provided membrane.

In some embodiments, the catalyst is associated with or attached to a substrate surface that allows a large surface area for the hydrogen gas to dissolve as molecular hydrogen into the process medium at an enhanced rate.

In some embodiments, the redox enzyme or enzymes consuming the reduced form of the cofactor are positioned within the chamber or vessel in such a manner that they are within 1 micron, 2 microns, 5 microns, 10 microns, 20 microns, 50 microns, 100 microns or 200 microns from the hydrogenase enzyme.

In some embodiments, the system further includes any enzyme for converting the 1,2-NAD(P)H₂, 1,6-NAD(P)H₂, and/or [NAD(P)]₂ into the second recovered NAD(P).

In some embodiments, the system further includes the renalase enzyme and/or the Mung Bean Phenol Oxidase enzyme for converting the 1,2-NAD(P)H₂, 1,6-NAD(P)H₂, and/or [NAD(P)]₂ into the second recovered NAD(P).

In some embodiments, the system further includes catalase for decomposing hydrogen peroxide produced by the renalase enzyme, the Mung Bean Phenol Oxidase enzyme.

Also provided herein is a system for providing NAD(P)H₂ reducing equivalents to an in vitro or cell-free system, comprising:
an electrochemical cell comprising an anode contained in an anode chamber and a cathode contained in a cathode chamber;
deionized water in the anode chamber in contact with the anode;
a proton permeable membrane that separates the anode and cathode chambers;
a cathode, optionally constructed of porous material capable of allowing convective flow of the process stream through the geometric volume of the cathode;
a liquid phase in the cathode chamber continuously in contact with the cathode, said liquid phase containing the cofactor required for the desired redox reaction;
optionally, a catalyst which acts upon the hydrogen formed at the cathode to reduce the oxidized form of the cofactor, thus forming the desired reduced form of the cofactor, such catalyst being capable of accepting hydrogen molecules formed at the cathode and catalyzing the reduction of the oxidized form of the cofactor;
optionally, attachment of the catalyst to the cathode surface by physical or chemical methods;
a process stream containing a substrate to be transformed via catalysis by the redox enzyme system which oxidizes the reduced from the cofactor with concomitant production of a desired product;
optionally, a membrane located between the cathode and the process stream, said membrane capable of preventing the optional catalyst from significantly leaving the cathode chamber and entering into the process stream;
optionally, the cathode constructed of porous material capable of allowing convective flow of the process stream through its geometric volume;
optionally, containment within the cathode chamber of any of the components of the system, the cofactor in either oxidized or reduced forms, the optional catalyst, and the redox system utilizing the reduced from of the cofactor;
an external power source providing a voltage between the anode and the cathode, and capable of controlling the voltage applied between the anode and the cathode, and the current provided, such that the voltage and the current may be controlled in order to prevent or enhance the formation of hydrogen at the cathode, and thus prevent or enhance the production of bulk amount of hydrogen gas formed in the cathode chamber.

In some embodiments, the cathode comprises porous or foamed carbon.

In some embodiments, the cathode comprises a porous structure of sintered or compressed metal particles.

In some embodiments, the cathode comprises a polymer formed as an open cell foam and capable of acting as a cathode itself, or covered with a material capable of acting as a cathode.

In some embodiments, the cathode comprises a porous ceramic capable of acting as a cathode itself, or covered with a material capable of acting as a cathode.

In some embodiments, the catalyst is capable of accepting hydrogen molecules formed at the cathode in the liquid phase prior to the formation of hydrogen bubbles.

In some embodiments, the catalyst is a hydrogenase enzyme.

In some embodiments, a chemical compound capable of using molecular hydrogen to reduce the oxidized form of the cofactor

In some embodiments, the system further includes attachment of the catalyst to the cathode surface by physical or chemical methods.

In some embodiments, the system further includes containment of the catalyst in the porous structure of the cathode.

In some embodiments, the system further includes containment of the in vitro or cell free-system within the cathode chamber.

In some embodiments, the system further includes renalase enzyme and the Mung Bean Phenol Oxidase enzyme for converting the 1,2-NAD(P)H₂, 1,6-NAD(P)H₂, and/or [NAD(P)]₂ into the second recovered NAD(P).

In some embodiments, the system further includes catalase for decomposing hydrogen peroxide produced by the renalase enzyme, the Mung Bean Phenol Oxidase enzyme.

In some embodiments, the system further includes a process stream containing a substrate to be transformed via catalysis by the redox enzyme system which oxidizes the reduced from the cofactor with concomitant production of a desired product.

In some embodiments, the redox enzyme or enzymes consuming the reduced form of the cofactor are positioned within the cathode chamber in such a manner that they are within 1 micron, 2 microns, 5 microns, 10 microns, 20 microns, 50 microns, 100 microns or 200 microns from the hydrogenase enzyme.

A further aspect relates to a cathode, comprising a porous carbon material and a hydrogenase associated therewith, wherein the hydrogenase is optionally incorporated into and/or attached onto the porous carbon material.

The present disclosure, in one aspect, comprises a method to provide electrons, presented as molecular hydrogen, to the oxidized cofactor NAD, thus transforming it to its reduced state NADH₂ by the action of a catalyst, for use by redox or other enzymes in an in vitro or cell free system, for example, the enzyme systems disclosed in U.S. Ser. No. 10/696,988B2, “Electrochemical Bioreactor Module and Engineered Metabolic Pathways for 1-Butanol Production with High Carbon Efficiency”, and PCT publication No. WO2020167999A1, “Cell-Free Compositions for ATP Regeneration and Uses Thereof”.

The disclosed process may, optionally, include steps for preventing the loss of cofactor to the reduced tautomers 1,2-NAD(P)H₂ and 1,6-NAD(P)H₂ and the dimer [NAD(P)]₂ that are not useful to redox enzymes as taught in PCT publication No. WO2017160793A1, “Improved Method for Using Electrochemical Bioreactor Module with Recovery of Cofactor”.

In its most general form, the process comprises the introduction of hydrogen into a chamber containing a catalyst, and through which an aqueous stream of NAD(P) is passing such that the NAD(P) is reduced to NAD(P)H₂ by the catalyst, the NAD(P)H₂ being made available for use by redox enzymes, or other enzymes requiring reducing equivalents. Arrangements are made to increase the rate of reduction of NAD(P) to NAD(P)H₂ to levels sufficient for commercial use. More specifically, the disclosed process uses hydrogenase enzyme as the catalyst to reduce NAD(P) to NAD(P)H₂ with the concomitant consumption of hydrogen.

### DETAILED DESCRIPTION

There are a variety of cofactors used in biological systems, such as flavin mononucleotide (FMN), various quinone species, etc., but the most common cofactor is nicotinamide adenine dinucleotide. A phosphorylated form of the cofactor, also exists, and both forms provide reducing equivalents to the enzymes that catalyze reactions requiring reducing equivalents. Other cofactors are well known, and also mediate electron transport in biological systems. Such cofactors include flavin mononucleotide (FMN), ubiquinone, cytochromes, and components of the electron transport chain (ETC) found, for example, in the mitochondria.

The non-phosphorylated and phosphorylated forms of the nicotinamide adenine dinucleotide cofactors both exist in an oxidized state and in a reduced state, and the current literature most commonly uses the descriptor “NAD(P)+” to indicate the oxidized state of the phosphorylated and non-phosphorylated forms of the nicotinamide adenine dinucleotide, and “NAD(P)H” to indicate the reduced state. However, these abbreviations will be avoided here and simplified to NAD(P) and NAD(P)H₂ respectively. The reduced state of the phosphorylated and non-phosphorylated forms of the nicotinamide adenine dinucleotide are produced by adding two electrons and two protons, (formally, one hydrogen molecule, H₂), to the oxidized state. Thus, the common nomenclature is misleading, as plain reading of the common descriptors “NAD(P)+” and “NAD(P)H” show the oxidized state of the phosphorylated and non-phosphorylated forms of the nicotinamide adenine dinucleotide to be a positively charged species “NAD(P)+”, while the reduced state of the phosphorylated and non-phosphorylated forms of the nicotinamide adenine dinucleotide, “NAD(P)H”, is shown to have only one hydrogen added relative to the oxidized state. Neither of these representations is true. The oxidized state of the phosphorylated and non-phosphorylated forms of the nicotinamide adenine dinucleotide is a neutral molecule in which the nitrogen of the nicotinamide ring bears a formal positive charge with is balanced by a negative charge one of the deprotonated phosphate linkages, thus forming an internal salt or zwitterion. The reduced state of the phosphorylated and non-phosphorylated forms of the nicotinamide adenine dinucleotide has formally accepted a hydrogen molecule, H₂, relative to the oxidized state. This is shown formally by the following balanced chemical reaction:

NAD(P)+H₂→NAD(P)H₂

The published molecular weights of the oxidized and reduced states of the non-phosphorylated form of the nicotinamide adenine dinucleotide indicate the reality of this reaction clearly, the molecular weights being 663.43 Da and 665.44 Da respectively and the difference being the molecular weight of a single hydrogen molecule, H₂.

In the present disclosure, the descriptor “NAD(P)” indicates the oxidized state of the phosphorylated and non-phosphorylated forms of the nicotinamide adenine dinucleotide, while the descriptor “NAD(P)H₂” indicates the reduced state of the phosphorylated and non-phosphorylated forms of the nicotinamide adenine dinucleotide. Further, the descriptors “1,2-NAD(P)H₂”, “1,4-NAD(P)H₂” and “1,6-NAD(P)H₂” are used to indicate the three stereoisomers of the nicotinamide ring that can occur upon reduction of NAD(P), in which a hydrogen molecule has been formally added across the 1,2-, 1,4- and 1,6-positions of the nicotinamide ring. It will be clear to those skilled in the art that 1,4-NAD(P)H₂ is commonly called β-NADH, and is the stereoisomer of the reduced state of the phosphorylated and non-phosphorylated forms of nicotinamide adenine dinucleotide which is active with oxidoreductases, P450 enzymes and other components of electron transport systems.

In the present disclosure, the 4,4′-dimer that is also known to form as a consequence of a one-electron transfer to NAD(P) is indicated by the descriptor “[NAD(P)]₂”.

Most generally, the enzymes accepting reducing equivalents from the reduced state of the nicotinamide adenine dinucleotide cofactors are termed “oxidoreductases” and are classified by the Nomenclature Committee of the International Union of Biochemistry and Molecular Biology as EC 1.n.n.n. Of particular interest are the enzymes generally termed “dehydrogenases” or “ketoreductases” in the classes EC 1.1.n.n and EC 1.2.n.n, as well as those termed “mono-oxygenases” in the classes EC1.13.n.n and EC.1.14.n.n. This last group of enzymes are also termed “P450” enzymes or “CYP” enzymes.

When performing reactions catalyzed by such enzymes using micro-organisms growing on a carbon source such as a carbohydrate, the reducing equivalents are generated by oxidizing a portion of the carbohydrate to CO₂, that is, some of the carbohydrate provided to the micro-organism is sacrificially oxidized in order to provide reducing equivalents for the micro-organism to use in various metabolic processes. While the resulting reducing equivalents are useful to the microorganism, the carbon atoms sacrificed by oxidation of the input carbon source to CO₂ are lost. It is also possible to provide carbon sources other than carbohydrates that are also sacrificed to produce the desired reducing equivalents. It will be clear that while such an in vivo process may perform the desired reaction catalyzed by the oxidoreductase enzyme, recovering the desired product from the milieu of the in vivo system, e.g. a fermentation broth, can be difficult. It is thus advantageous to utilize an in vitro system for performing the desired reaction.

It is possible to isolate the needed oxidoreductase enzyme and use it to catalyze reactions in vitro. Significantly improved chemical processes could be achieved by an in vitro system which allows the use of the plethora of oxidoreductase enzymes in processes resembling standard catalytic chemical processes. Such systems avoid the issues of recovering the desired product of the reaction from fermentation broths, and can provide further advantages by allowing the enzyme to be used under non-physiological conditions, and the use of a variety of methods to immobilize or otherwise contain the enzyme, and allow it to be used for extended periods of time. Such immobilization or other containment methods also allow simple and efficient recovery of the desired product of the reaction from the enzyme system.

A further advantage of using isolated enzymes in vitro is that multiple enzymes can be used, allowing a series of reactions to be performed in tandem to transform an input reagent, such as a carbohydrate, to a desired final product. This arrangement is generally termed cell-free synthesis, and is now recognized as a powerful method for producing valuable commercial compounds via biological processes, without the need for practicing classical fermentation.

However, the reducing equivalents must still be provided to such in vitro or cell-free systems if there are reduction steps required to transform the input starting material to the desired product. Analogous to in vivo systems, a sacrificial substrate can be provided, which is oxidized. This generates the necessary reduced state of the cofactors for providing reducing equivalents for the desired reaction. In cell-free systems, larger collections of enzymes can be used which catalyze portions of pathways occurring in in vivo systems which oxidize the input starting material to generate the necessary reduced cofactors.

It will be clear that the need of a sacrificial substrate removes at least some of the advantages of an in vitro system, since a second product, the product of the oxidation of the sacrificial substrate, must now be separated from the desired product. In the case of in vitro systems incorporating several enzyme reactions, or cell free systems, input glucose can also be oxidized to provide the desired reducing equivalents, but now the problem of consuming input starting material to produce reducing equivalents prevents full incorporation of the input starting into the desired final product, and thus suffers from the same inefficiency and reduction of mass yield and as in vivo systems, such as classical fermentations. Without the use of an external source of reducing power, some amount of the input carbon (e.g. carbohydrate) must generally be oxidized to generate reducing equivalents required elsewhere in the overall system. This removes carbon available for product formation and lowers product yields.

Thus it is most desirable to provide the reducing equivalents required for any reduction reactions present in the system, that is, the reducing equivalents required by the oxidoreductase enzyme or enzymes present in the system, in a manner that does not require sacrificial substrates with the concomitant production of by-products, and does not consume input starting material in a manner which prevents it from being incorporated into the final product and reducing overall mass yield of the process.

If electrons comprising the reducing equivalents could be provided from an external source, that is, an electrical current, then the need to provide a sacrificial substrate to provide the reducing equivalents would be eliminated, and oxidoreductase enzymes could be used as conventional catalysts, performing reactions without the need for living cells or associated enzyme systems or processes.

This situation has been recognized by others, and a number of attempts to deliver electrons to biological systems by electrochemical methods have been published. To date, these have not resulted in any practical systems that can be used for producing chemicals at large scale.

It has been reported by Park and Zeikus in J. Bacteriol. 181:2403-2410, 1999 that the compound called Neutral Red would undergo reversible chemical oxidoreductions with the nicotinamide adenine dinucleotide cofactor, that is, Neutral Red in its reduced form (NR_{red}) has a sufficiently low redox value that it will transfer electrons to, and thus reduce, the nicotinamide adenine dinucleotide cofactor from its oxidized state to its reduced state. In this process, the Neutral Red becomes oxidized to the species NRₒₓ which is then available to accept an electron from the cathode and thus return to the reduced form NR_{red}, which is in turn available to again reduce the oxidized state of the nicotinamide adenine dinucleotide cofactor.

U.S. Pat. No. 7,250,288 B2 to Zeikus et al. discusses the need for improving electrode efficiencies in electrochemical bioreactor system and proposes improvements such as linking the nicotinamide adenine dinucleotide cofactor, Neutral Red, and the enzyme fumarate reductase to the electrode in order to improve electron transfer characteristics. While the above may improve electron transfer characteristics, it may also be advantageous to improve upon the electrochemical bioreactor system design and its use in other ways such as those described below.

Other methods of introducing electrons from external sources are also possible, most obviously the introduction of hydrogen gas. Hydrogen gas can be used in systems combining enzymes and non-biological catalysts, termed chemo-enzymatic systems, that can utilize the hydrogen gas to reduce enzyme cofactors (“Efficient Catalytic Interconversion between NADH and NAD+ Accompanied by Generation and Consumption of Hydrogen with a Water-Soluble Iridium Complex at Ambient Pressure and Temperature”, Yuta Maenaka, Tomoyoshi Suenobu, Shunichi Fukuzumi, J. Am. Chem. Soc. 2012, 134, 1, 367-374, doi.org/10.1021/ja207785f).

A group of enzymes known as hydrogenases are capable of catalyzing the reversible transfer of electrons between cofactors, thus mediating the formation of hydrogen gas from NADH₂ (leaving NAD), or the reduction of NAD to NADH₂ with the consumption of molecular hydrogen, i.e., H₂, most commonly presented as hydrogen gas.

In the case of either the chemo-enzymatic method or the method utilizing hydrogenases, the hydrogen gas may be delivered from any source, including an electrochemical cell performing the electrolysis of water.

Hydrogenases occur in several distinct classes, but the most common hydrogenases are prototypical [FeFe]-hydrogenases which contain two iron atoms bonded by a sulfur bridge at their active site. These perform hydrogen turnover using ferredoxin as a redox partner, while bifurcating versions of hydrogenases perform the same reaction using both ferredoxin and NAD(P) as electron donor or acceptor. (Schuchmann, Kai; Chowdhury, Nilanjan Pal; Müller, Volker (2018-12-04). “Complex Multimeric [FeFe] Hydrogenases: Biochemistry, Physiology and New Opportunities for the Hydrogen Economy”. Frontiers in Microbiology. 9. doi:10.3389/fmicb.2018.02911).

An example of the best-characterized and catalytically most active enzymes are the [FeFe]-hydrogenase from Chlamydomonas reinhardtii (CrHydA1), Thomas; NABER, J. Dirk (June 1993). “Isolation, characterization and N-terminal amino acid sequence of hydrogenase from the green alga Chlamydomonas reinhardtii”. European Journal of Biochemistry. 214 (2): 475-481. doi:10.1111/j.1432-1033.1993.tb17944.x) Desulfovibrio desulfuricans (DdHydAB or DdH) (Glick, Bernard R.; Martin, William G.; Martin, Stanley M. (1980-10-01). “Purification and properties of the periplasmic hydrogenase from Desulfovibrio desulfuricans”. Canadian Journal of Microbiology. 26 (10): 1214-1223. doi:10.1139/m80-203) and Clostridium pasteurianum and Clostridium acetobutylicum (CpHydA1 and CaHydA1), (Nakos, George; Mortenson, Leonard (March 1971). “Purification and properties of hydrogenase, an iron sulfur protein, from Clostridium pasteurianum W5”. Biochimica et Biophysica Acta (BBA)—Enzymology. 227 (3): 576-583. doi:10.1016/0005-2744(71)90008-8).

The preparation of hydrogenases has been described in the open literature, for example, Sun J, Hopkins R C, Jenney F E Jr, McTernan P M, Adams M W W (2010) Heterologous Expression and Maturation of an NADP-Dependent [NiFe]-Hydrogenase: A Key Enzyme in Biofuel Production. PLoS ONE 5(5): e10526. doi:10.1371/journal.pone.0010526; Qin Fan, Peter Neubauer, Oliver Lenz, Matthias Gimpel (2020), Heterologous Hydrogenase Overproduction Systems for Biotechnology—An Overview, Int. J. Mol. Sci. 2020, 21, 5890; doi:10.3390/ijms21165890; all of which are incorporated herein by reference.

The requirements for providing reducing equivalents by electrochemical methods are disclosed in PCT publication No. WO2014039767 A1, “Electrochemical Bioreactor Module and Methods of Using the Same”. The use of isolated enzymes in conjunction with the electrochemical reduction of the oxidized form of nicotinamide adenine dinucleotide cofactor to its reduced form, and considerations to be made, are disclosed in PCT publication No. WO2016070168 A1, “Improved Electrochemical Bioreactor Module and Use Thereof”. The recovery of the undesired tautomers of NADH₂, and the dimer of NAD, are disclosed in PCT publication No. WO2017160793A1, “Improved Method for Using Electrochemical Bioreactor Module with Recovery of Cofactor”.

Regardless of the method used to deliver exogenous electrons and the necessary balancing protons (e.g. as molecular hydrogen, H₂O), the rate of delivery of such electrons must be high enough to sustain a useful rate of production of the desired final material via the redox reaction or reactions performed by the in vitro or cell free system, and to do so in a convenient manner. This further requires a sufficient concentration of cofactor molecules to accept the exogenous reducing equivalents that a sufficient number of reducing equivalents can be transported from the source (e.g. the surface of an electrode, or the surface of a gas phase of hydrogen) to the redox enzyme or enzymes in a sufficiently short period of time, that the overall reaction rate of the redox enzyme or enzymes is sufficiently high to permit the useful production of the desired material by the in vitro or cell free system.

Even at extreme concentrations, it is difficult at large scale to provide a process stream of a solution of NAD passing through an arrangement of hydrogenase enzymes mediating the reduction of NAD to NADH₂ in the presence of hydrogen gas, such that a sufficient numbers of NAD molecules are reduced by the hydrogenase via the consumption of hydrogen gas, exit the part of the system containing the hydrogenases to which hydrogen gas is being fed, deliver the reducing equivalents to a redox enzyme in the in vitro or cell-free system, and return to the hydrogenase enzymes in a sufficiently short time to allow a rate of transfer of reducing equivalents from the hydrogen gas to the enzyme-catalyzed reaction or reactions in the in vitro or cell free system to allow the system to produce the final product at a useful rate.

For example, if a molecule of NADH₂ is formed by the reaction of molecular hydrogen via a hydrogenase, that molecule of NADH₂ must then move to the redox enzyme in order to by utilized, and that physical movement requires some amount of time. If the molecule of NADH requires one second to traverse the physical volume of the system to reach the redox enzyme, then be oxidized to NAD, and then return to the hydrogenase to be reduced back to NADH, then the redox enzyme is restricted to performing one reaction every two seconds. If the redox enzyme is capable of oxidizing 1,000 molecules of NADH to NAD each second, then 2,000 molecules of nicotinamide adenine dinucleotide cofactor would be required to move reducing equivalents from the hydrogenase to the redox enzyme in order to allow the redox enzyme to operate at its maximum rate. If it were to require only a millisecond for the NADH produced by the hydrogenase to reach the redox enzyme, then only two molecules of nicotinamide adenine dinucleotide cofactor would be required to allow the redox enzyme to operate at its maximum rate.

Further, hydrogen gas must dissolve in the aqueous process stream containing the NAD and the hydrogenase. The consensus model of the mechanism of the hydrogenase enzymes requires that molecular hydrogen, i.e. H₂, be added to the iron-sulfur centers of the enzyme. The solubility of molecular hydrogen in water is very low, and this forces another constraint on the use of hydrogenases and hydrogen gas as a method of reducing NAD to NADH₂ at a rate sufficient to be useful at commercial scale. While the system could be pressurized to increase the solubility of the hydrogen gas in the aqueous process stream containing NAD and contacting the hydrogenase enzyme, very high pressures would be needed to appreciably increase the concentration of dissolved hydrogen. An additional constraint of such of such a system for regenerating NADH₂ is the hazardous nature of hydrogen gas, especially under pressure.

As a non-limiting example, the production of succinic acid from CO₂ requires 7 moles of NADH₂ for every mole of succinic acid produced.

4CO₂+7H₂→C₄H₆O₄+4H₂O

With a molecular weight of 118 for succinic acid, the production of 11.8 grams per hour per liter of reactor volume requires the delivery of 0.7 moles of NADH₂ to that same reactor volume of one liter every hour. With a molecular weight of 665 for NADH₂, that requires 4,650 grams of NADH₂ to be delivered each hour of operation to that one liter of reactor volume if each molecule of NADH₂ delivers one reducing equivalent in that hour. If the total volume of the process stream passing through the various pipes etc. needed to reach that one liter of reactor volume is itself 10 liters in volume, then approximately 46,500 grams of NADH would need to be present in the entire process stream. If NADH₂ can be oxidized to NAD by the redox enzymes at a rate of 1,000,000 times per hour (i.e. approximately 280 times per second), then only 93 milligrams of the cofactor (combined amounts of NADH going to the redox enzymes and NAD returning to the hydrogenase) should be required in the entire process stream to allow the redox enzymes to work at maximum rate. However, the entire contents of the process stream would have to recirculate 1,000,000 times during that hour (280 times per second), in order for the 93 milligrams of the cofactor to move sufficiently quickly between the hydrogenase enzyme and the redox enzymes to allow the production of 11.8 grams of succinic acid each hour in the one liter reactor.

A solution to the practical issue of such high speed recirculation is to reduce the volume of the system. For example, if the distance from the hydrogenase enzyme producing the NADH to the redox enzyme consuming the NADH₂ were on the order of 1 micron, then diffusion alone would allow the NADH₂ molecule to move between the hydrogenase and the redox enzyme in approximately 1 millisecond. Thus, with a physical spatial arrangement positioning the hydrogenase and the necessary source of molecular hydrogen approximately 1 micron from the redox enzyme or enzymes comprising an in vitro or cell free redox system, one molecule of nicotinamide adenine dinucleotide cofactor could move from the hydrogenase (as NADH₂) to a redox enzyme to deliver the reducing equivalent it carries, and return (as NAD) to the hydrogenase 500 times in one second, and significantly lowering the total amount of nicotinamide adenine dinucleotide cofactor required in a production system operating at a commercially useful scale.

Provided herein are methods and systems for achieving the desired objective of transferring externally provided electrons to NAD, in order to produce NADH₂ and provide the produced NADH₂ to redox enzyme or enzymes at rates sufficiently high to allow the production of commercially desirable compounds by in vitro enzymatic or cell free systems requiring reducing equivalents. Other objectives, features, and advantages of the present disclosure will be apparent on review of the specification and claims.

The present disclosure, in some embodiments, is directed to an improved version and improved use of the “Electrochemical Bioreactor Module” (EBM) previously described in PCT Patent Application Publication Nos. WO2014039767 A1 and WO2016070168 A1, and U.S. Ser. No. 10/696,988B2, “Electrochemical Bioreactor Module and Engineered Metabolic Pathways for 1-Butanol Production with High Carbon Efficiency”, and PCT publication No. WO2020167999A1, “Cell-Free Compositions for ATP Regeneration and Uses Thereof”, which are incorporated herein by reference in their entirety.

Hydrogen molecules for reducing the oxidized form the of cofactor can be provided by hydrogen gas from an external source, without the use of an electrochemical cell to generate hydrogen molecules in close proximity to the catalyst facilitating the reducing of the oxidized form of the cofactor. In such an embodiment, the hydrogen is most optimally provided in aqueous solution of dissolved hydrogen molecules, as this removes the physical process of transferring hydrogen from the gas phase, across a phase boundary, and dissolving into the aqueous medium.

In one embodiment, the method for providing reducing equivalents can include one or more of the following components:
a. a chamber or vessel, capable of receiving hydrogen and capable of receiving or containing a liquid phase, said liquid phase containing the cofactor requiring reduction from its oxidized form to its reduced form, for use in the desired redox reaction or reactions performed by an in vitro or cell-free process, and;
b. a catalyst which enhances the rate of reduction of the oxidized form of the cofactor by the presented molecular hydrogen, thus forming the desired reduced form of the cofactor, such catalyst being, optionally, a hydrogenase enzyme capable of accepting molecular hydrogen and catalyzing the reduction of the oxidized form of the cofactor, and;
c. a process stream containing a substrate to be transformed via catalysis by the redox enzyme system which oxidizes the reduced from the cofactor with concomitant production of a desired product, and;
d. the structure of the chamber or vessel allowing the positioning of the catalyst immediately proximal to the source of molecular hydrogen such that the hydrogen is presented to the catalyst as molecular hydrogen dissolved in the process medium, and;
e. the redox enzyme or enzymes consuming the reduced form of the cofactor are positioned within the chamber or vessel in such a manner that they are within 1 micron, 2 microns, 5 microns, 10 microns, 20 microns, 50 microns, 100 microns or 200 microns from the catalyst, and;
f. optionally, the catalyst is associated with or attached to a surface that allows a large surface area for the hydrogen gas to dissolve as molecular hydrogen into the process medium at an enhanced rate, and;
g. optionally, a membrane or membranes enabling containment within the chamber or vessel of any of the components of the system in any combination, the cofactor in either oxidized or reduced forms, the catalyst, and the process stream containing redox system utilizing the reduced from of the cofactor.

Hydrogen may be presented to the chamber or vessel in any convenient manner, but it is preferred to present the hydrogen dissolved in the liquid phase, and not as large bubbles of hydrogen gas. Large bubbles of hydrogen gas will require that the hydrogen molecules to cross the liquid/gas phase boundary and dissolve in the liquid phase, and this will reduce the rate of hydrogen delivery to the catalyst, and thus reduce the rate of the reduction of the cofactor. Large bubbles of hydrogen gas will also present safety considerations.

In one embodiment, hydrogen is presented using a membrane system. In this embodiment, to prevent the presence of large bubbles of hydrogen gas in the chamber or vessel, a membrane system with a large surface area contacting the liquid stream on the first side of the membrane and contacting hydrogen gas on the second side of the membrane, the composition of the membrane being such that molecular hydrogen can diffuse through the membrane and dissolve in the liquid stream. A catalyst capable of accepting the hydrogen and reducing the oxidized from of the cofactor provided in the liquid phase, is positioned either on, or close to, the first surface of the membrane, such that it acts upon hydrogen crossing the membrane and dissolving the liquid phase before the hydrogen can exceed its solubility limit and form bubbles. In this manner, the rate of cofactor reduction is maximized.

In another embodiment, hydrogen is presented as nanobubbles in the liquid phase. Nanobubbles are 200 nanometers or less in diameter, and have physical characteristics different from larger bubbles. Nanobubbles move with Brownian motion and do not readily coalesce to form larger bubbles. Further, as the ratio of the surface area of a nanobubble to its volume is much larger than the ratio of surface area to volume in a bubble of gas that is larger in diameter than 200 nanometers, the rate of transfer of the gas from the nanobubble into the liquid phase surrounding the nanobubble is much greater than for larger bubbles. In this manner, delivery of hydrogen dissolved in the liquid phase, to the catalyst performing the reduction of the cofactor, is maximized. Nanobubbles can be created using commercially available equipment (www.moleaer.com)

The electrolysis of water in an electrochemical cell generates hydrogen at the cathode surface by delivering electrons from the electrical circuit to the cathode, where they combine with protons (present in an aqueous medium as hydronium ions, H₃O⁺) to produce a hydrogen atom. These hydrogen atoms combine with each other to produce a hydrogen molecule, H₂. The hydrogen molecule is neutral, and diffuses away from the surface of the cathode, dissolved in the aqueous medium. As the concentration of H₂ dissolved in the aqueous medium increases, it reaches the solubility limit of hydrogen in that medium, and forms a gas bubble. These nascent gas bubbles can coalesce, forming larger bubbles, which then leave the area of the cathode as a stream of hydrogen gas.

If a catalyst capable of accepting hydrogen (and catalyzing the reduction of a cofactor) is positioned either on or sufficiently close to the cathode surface that it acts upon the hydrogen produced at the cathode before the hydrogen molecules exceed the solubility limit of hydrogen and form large gas bubbles and macroscopic gas phase, then the rate of transfer of electrons from the cathode, as molecular hydrogen, to the oxidized form of the cofactor requiring reduction of the cofactor, will be maximized.

It is recognized that hydrogen molecules forming at the cathode will dissolve in the process medium, but may also form nanobubbles, that is, gas bubbles that are not larger than 200 nanometers in diameter. Thus, it is clear that electrolysis of water may provide a source of hydrogen nanobubbles as mentioned in the previously described embodiment.

In another embodiment, the method for providing reducing equivalents can incorporate an electrochemical cell for the production of hydrogen, and will include one or more of the following components:
a. an electrochemical cell comprising an anode contained in an anode chamber and a cathode contained in a cathode chamber, and;
b. deionized water in the anode chamber in contact with the anode, and;
c. a proton permeable membrane that separates the anode and cathode chambers, and;
d. a liquid phase in the cathode chamber continuously in contact with the cathode, said liquid phase containing the cofactor required for the desired redox reaction, and optionally, capable of being recirculated through the cathode chamber, and;
e. a cathode, optionally constructed of porous material capable of allowing convective flow of the process stream through its geometric volume, such a material being a vitreous carbon foam, a sintered metal, a polymer formed as an open cell foam and capable of acting as a cathode itself, or covered with a material capable of acting as a cathode, or a porous ceramic capable of acting as a cathode itself, or covered with a material capable of acting as a cathode, and;
f. a catalyst which acts upon the hydrogen formed at the cathode surface to reduce the oxidized form of the cofactor, thus forming the desired reduced form of the cofactor, such catalyst being, for example, a hydrogenase enzyme capable of accepting hydrogen molecules formed at the cathode and catalyzing the reduction of the oxidized form of the cofactor, or a chemical compound capable of using molecular hydrogen to reduce the oxidized form of the cofactor, and;
g. optionally, attachment of the catalyst to the cathode surface by physical or chemical methods, and;
h. a process stream containing a substrate to be transformed via catalysis by the redox enzyme system which oxidizes the reduced from the cofactor with concomitant production of a desired product, and;
i. optionally, a membrane located between the cathode and the process stream, said membrane capable of preventing the optional catalyst from significantly leaving the cathode chamber and entering into the process stream, and;
j. optionally, containment within or the of any of the components of the system, the cofactor in either oxidized or reduced forms, the optional catalyst, and the redox system utilizing the reduced from of the cofactor, and;
k. an external power source providing a voltage between the anode and the cathode, and capable of controlling the voltage applied between the anode and the cathode, and the current provided, such that the voltage and the current may be controlled in order to prevent or enhance the formation of hydrogen at the cathode, and thus prevent or enhance the production of bulk amount of hydrogen gas formed in the cathode chamber.

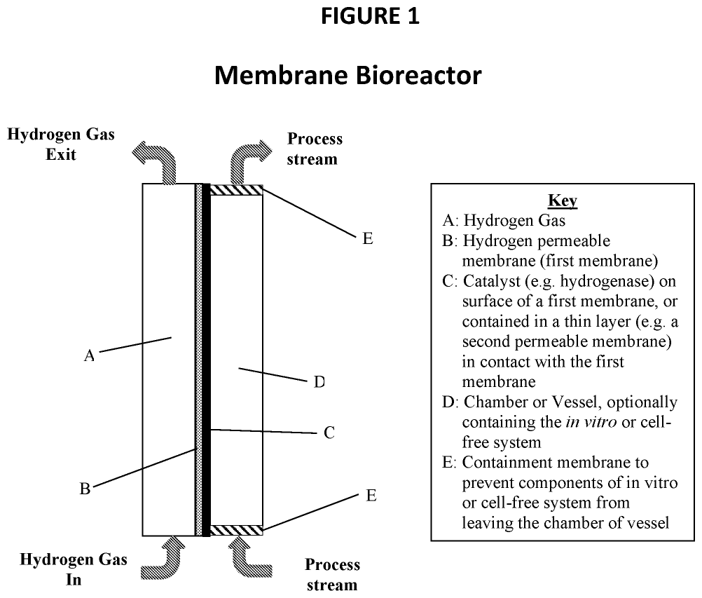

FIG. 1 illustrates, in a schematic, a system of providing a chamber or vessel, in which hydrogen gas is presented on the first side of a membrane, with a catalyst on the second side of the membrane, enabling the reduction of the cofactor, the cofactor being present in a process stream that enters chamber or vessel on the second side of the membrane.

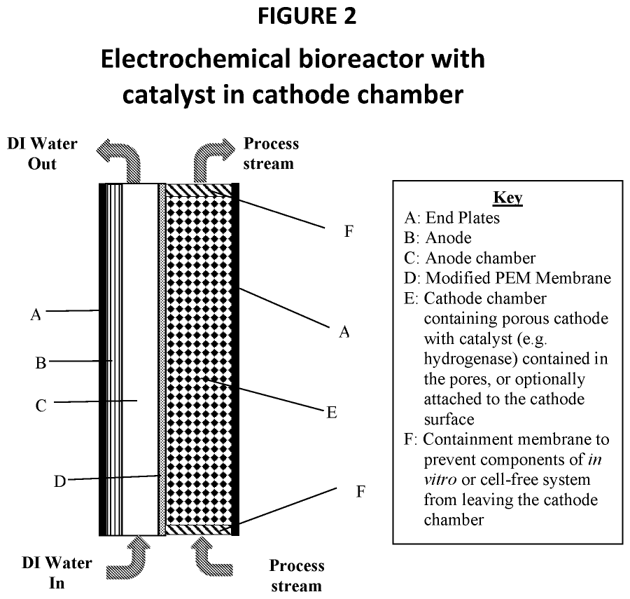

FIG. 2 illustrates, in a schematic, a system of providing hydrogen by means of water electrolysis, with a catalyst enabling the reduction of the cofactor using the newly formed hydrogen, such catalyst being attached to the surface of the cathode, or contained in the porous structure of the cathode.

It will be clear to those cognizant of enzymatic and electrochemical processes, that it is possible to dissolve hydrogen and utilize enzymes and other catalysts in non-aqueous media, and that non-aqueous media can also be passed through the anode and cathode chambers of an electrochemical cell.

In certain embodiments, processes of the present disclosure can include the enzyme renalase, the Mung Bean Phenol Oxidase, and illumination to recover the undesired forms of cofactor that result from reduction of oxidized form of the cofactor, and from the tautomerization of the reduced from of the cofactor itself, as disclosed in PCT publication No. WO2017160793A1, “Improved Method for Using Electrochemical Bioreactor Module with Recovery of Cofactor”.

Example: The non-catalyzed NAD+ reduction to NADH in a cell-free reaction chamber completely filled with a porous, conductive carbon block with recirculating process stream.

### Experimental Setup

A highly instrumented electrochemical bioreactor system with a PEM (DuPont, Nafion membrane, no catalyst) electrolyzer modified with a cathode chamber 15 cm wide, 15 cm high and 2.5 cm deep filled with a porous (porous conductive carbon, 20 pores per inch/high flow, purchased from McMaster-Carr), conductive carbon block was used to reduce NAD+. Two liters of pH 7.7 phosphate buffer was purged with nitrogen to remove traces of dissolved oxygen prior to adding 20 g of NAD. The NAD solution was recirculated at 30 ml/min through the porous cathode while deionized water was recirculated through the 14 ml anode chamber at a flowrate of 900 ml/min while the entire electrolyzer temperature was controlled at 23 degrees C. The flow through the porous cathode was monitored with ten on-line continuous UV detectors at 254 nm, 313 nm, 340 nm, 365 nm and 400 nm on the electrolyzer inlet and outlet, respectively. The power supply running the electrolyzer cell was used to control the voltage and continuously monitor the current delivered. pH probes were in-line before and after the cathode chamber while in-line temperature and conductivity sensors were in place before and after both the cathode chamber and anode chambers. The total oxygen produced in the anode chamber and the total hydrogen produced at the cathode were collected in each electrolyzer exit stream, respectively, and continuously measured to calculate the Faradaic efficiency and close the material balance around the system.

### Operating Conditions

The run begins with the voltage at 4.5V, after 30 min the voltage is adjusted to 2.8V. After another 3 hours the voltage is adjusted to 3.2V where it remains until the end of the 25-hour run. During each of these periods, the rate of change in 340 OD/hr is determined. From previous calibration work, the concentration of 1,4 NADH is 103.7 mg/1/OD. Using this information, the following table is generated to calculate the NADH produced and the NAD+ utilized during the 25-hr period.

### Results

The table below gives the amounts of NADH calculated from the detectors:

| Time (hrs) | Voltage (V) | NADH (mg) |
|---|---|---|
| 0.0-0.5 | 4.5 | 52 |
| 0.5-3.5 | 2.8 | 311 |
| 3.5-25.0 | 3.2 | 1468 |

### CONCLUSION

The data demonstrate the ability for the cell-free EBR System to control the NAD⁺ reduction rate based upon the operating conditions. Depending upon the desired cell-free product, the EBR System can regulate the NADH oxidation rate by manipulating the cathode temperature, the flowrate to the cathode chamber, the concentration of NAD⁺ and the voltage across the cathode chamber. Subsequent runs with a PtRu catalyst in the cathode chamber to facilitate hydrogen gas formation resulted in extremely low levels of NADH. Thus, an enzyme catalyst to promote NADH formation can improve the overall operation.

### EQUIVALENTS

The present disclosure provides among other things novel methods and devices for providing reducing equivalents to biological systems. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and patent applications cited above are incorporated by reference herein in their entirety for all purposes to the same extent as if each individual publication or patent application were specifically indicated to be so incorporated by reference.

1. A system for providing NAD(P)H₂ reducing equivalents to an in vitro system, comprising:
a. a chamber configured to receive hydrogen and to contain a liquid phase, said liquid phase comprising a cofactor that can be reduced by hydrogen from an oxidized form to a reduced form, for use in a desired redox reaction performed by an in vitro process;
b. a catalyst which enhances a reduction rate of the oxidized form by the hydrogen, thus forming the reduced form;
c. a process stream comprising a substrate to be transformed via the desired redox reaction which oxidizes the reduced form of the cofactor with concomitant production of a desired product; and
d. optionally, a membrane enabling containment within the chamber, the cofactor in the oxidized and/or reduced forms, the catalyst, and the process stream.

2. The system of claim 1, in which the catalyst is a hydrogenase enzyme.

3. The system of claim 1, comprising attachment of the catalyst to the membrane surface by physical or chemical methods.

4. The system of claim 1, in which the structure of the chamber or vessel allowing the positioning of the catalyst immediately proximal to the source of molecular hydrogen such that the hydrogen is presented to the catalyst as molecular hydrogen dissolved in the process medium.

5. The system of claim 1, comprising containment of the catalyst in the optionally provided membrane.

6. The system of claim 1, in which the catalyst is associated with or attached to a substrate surface that allows a large surface area for the hydrogen gas to dissolve as molecular hydrogen into the process medium at an enhanced rate.

7. The system of claim 2, in which the redox enzyme or enzymes consuming the reduced form of the cofactor are positioned within the chamber or vessel in such a manner that they are within 1 micron, 2 microns, 5 microns, 10 microns, 20 microns, 50 microns, 100 microns or 200 microns from the hydrogenase enzyme.

8. The system of claim 1, additionally comprising any enzyme for converting the 1,2-NAD(P)H₂, 1,6-NAD(P)H₂, and/or [NAD(P)]₂ into the second recovered NAD(P).

9. The system of claim 8, additionally comprising the renalase enzyme and/or the Mung Bean Phenol Oxidase enzyme for converting the 1,2-NAD(P)H₂, 1,6-NAD(P)H₂, and/or [NAD(P)]₂ into the second recovered NAD(P).

10. The system of claim 8, further comprising catalase for decomposing hydrogen peroxide produced by the renalase enzyme, the Mung Bean Phenol Oxidase enzyme.

11. A system for providing NAD(P)H₂ reducing equivalents to an in vitro or cell-free system, comprising:
a. an electrochemical cell comprising an anode contained in an anode chamber and a cathode contained in a cathode chamber;
b. deionized water in the anode chamber in contact with the anode;
c. a proton permeable membrane that separates the anode and cathode chambers;
d. a cathode, optionally constructed of porous material capable of allowing convective flow of the process stream through the geometric volume of the cathode;
e. a liquid phase in the cathode chamber continuously in contact with the cathode, said liquid phase containing the cofactor required for the desired redox reaction;
f. optionally, a catalyst which acts upon the hydrogen formed at the cathode to reduce the oxidized form of the cofactor, thus forming the desired reduced form of the cofactor, such catalyst being capable of accepting hydrogen molecules formed at the cathode and catalyzing the reduction of the oxidized form of the cofactor;
g. optionally, attachment of the catalyst to the cathode surface by physical or chemical methods;
h. a process stream containing a substrate to be transformed via catalysis by the redox enzyme system which oxidizes the reduced from the cofactor with concomitant production of a desired product;
i. optionally, a membrane located between the cathode and the process stream, said membrane capable of preventing the optional catalyst from significantly leaving the cathode chamber and entering into the process stream;
j. optionally, the cathode constructed of porous material capable of allowing convective flow of the process stream through its geometric volume;
k. optionally, containment within the cathode chamber of any of the components of the system, the cofactor in either oxidized or reduced forms, the optional catalyst, and the redox system utilizing the reduced from of the cofactor;
l. an external power source providing a voltage between the anode and the cathode, and capable of controlling the voltage applied between the anode and the cathode, and the current provided, such that the voltage and the current may be controlled in order to prevent or enhance the formation of hydrogen at the cathode, and thus prevent or enhance the production of bulk amount of hydrogen gas formed in the cathode chamber.

12. The system of claim 11 in which the cathode comprises porous or foamed carbon.

13. The system of claim 11 in which the cathode comprises a porous structure of sintered or compressed metal particles.

14. The system of claim 11 in which the cathode comprises a polymer formed as an open cell foam and capable of acting as a cathode itself, or covered with a material capable of acting as a cathode.

15. The system of claim 11 in which the cathode comprises a porous ceramic capable of acting as a cathode itself, or covered with a material capable of acting as a cathode.

16. The system of claim 11 in which the catalyst is capable of accepting hydrogen molecules formed at the cathode in the liquid phase prior to the formation of hydrogen bubbles.

17. The system of claim 11, in which the catalyst is a hydrogenase enzyme.

18. The system of claim 11, in which a chemical compound capable of using molecular hydrogen to reduce the oxidized form of the cofactor

19. The system of claim 11, comprising attachment of the catalyst to the cathode surface by physical or chemical methods.

20. The system of claim 11, comprising containment of the catalyst in the porous structure of the cathode.

21. The system of claim 11, comprising containment of the in vitro or cell free-system within the cathode chamber.

22. The system of claim 11, additionally comprising renalase enzyme and the Mung Bean Phenol Oxidase enzyme for converting the 1,2-NAD(P)H₂, 1,6-NAD(P)H₂, and/or [NAD(P)]₂ into the second recovered NAD(P).

23. The system of claim 22, further comprising catalase for decomposing hydrogen peroxide produced by the renalase enzyme, the Mung Bean Phenol Oxidase enzyme.

24. The system of claim 11, additionally comprising a process stream containing a substrate to be transformed via catalysis by the redox enzyme system which oxidizes the reduced from the cofactor with concomitant production of a desired product.

25. The system of claim 17, in which the redox enzyme or enzymes consuming the reduced form of the cofactor are positioned within the cathode chamber in such a manner that they are within 1 micron, 2 microns, 5 microns, 10 microns, 20 microns, 50 microns, 100 microns or 200 microns from the hydrogenase enzyme.

26. A cathode, comprising a porous carbon material and a hydrogenase associated therewith, wherein the hydrogenase is optionally incorporated into and/or attached onto the porous carbon material.
